(19) United States
(12) Patent Application Publication
(10) Pub. No.: US 2010/0057049 A1
(43) Pub. Date: Mar. 4, 2010

(54) Multi-lumen catheter for the withdrawal of blood samples

(76) Inventor: Paul D. Levin, Santa Cruz, CA (US)

Correspondence Address: Bruce H. Johnsonbaugh;Eckhoff & Hoppe, 8th Floor, 1900 N. California Blvd., Walnut Creek, CA, 94596, US

(21) Appl. No.: 12/387,767

(22) Filed: May 7, 2009

Related U.S. Application Data
(63) Continuation-in-part of application No. 12/231,531, filed on Sep. 3, 2008.
Application No. 12/231,531 is a continuation-in-part of application No. 12/287,571, filed on Oct. 10, 2008.

(51) Int. Cl. A61M 25/00

(52) U.S. Cl. 604/523

(57) ABSTRACT

A multi-lumen catheter is described with features that reduce the volume of purge fluid needed when used with a system for monitoring blood analytes. The modifications involve reducing the internal diameter of one of the catheter tails, shortening its length, and reducing or eliminating the gap inside the Luer connector at the proximal end of the modified tail.

### CROSS-REFERENCE TO RELATED APPLICATIONS

This application is a Continuation-in-Part of U.S. application Ser. No. 12/231,531 filed on Sep. 3, 2008; and is also a Continuation-in-Part of U.S. application Ser. No. 12/287,571 filed on Oct. 10, 2008.

### BACKGROUND AND SUMMARY OF INVENTION

The majority of patients in a hospital ICU now have a central line for vascular access. Generally, central lines are used for infusions and only rarely are used for the withdrawal of blood samples. With the advent of tight glucose control, central lines now appear attractive for frequent blood sampling in addition to their use for the infusion of fluids containing electrolytes and various medications.

A system for intermittent blood sampling and testing for glucose is described in the above referenced U.S. patent application Ser. No. 12/231,531 by Levine Grage and Warner, incorporated herein by reference. In this system for frequent testing of blood glucose, about 0.2 to 0.4 mL of blood is withdrawn from the patient by a peristaltic pump, and then returned to the patient's circulation by pump reversal. It is important to clear the line completely after each test to avoid the mixing of successive samples. Two 500 fluid milliliter bags hang near the bedside monitor, and this fluid is used both to calibrate the sensor and to purge the line after each test for blood glucose. The Ser. No. 12/231,531 patent application describes methods to reduce the volume of purge fluid so as not to overload the patient's circulation with such fluid.

Potential problems involving a central venous catheter when used in conjunction with the glucose monitor were not addressed in the above mentioned patent application. It has been determined, however, that a standard venous catheter significantly increases the volume of purge fluid needed in such a system. Modifications of a standard triple lumen catheter to reduce the amount of purge fluid required are the subject of this application.

A primary object of the present invention is to provide a central venous catheter which allows the minimum amount of purge fluid to be used in a system of long term blood glucose monitoring.

A further object of the invention is to do so without reducing the utility of the catheter for the infusion of fluids.

A further object of the invention is to introduce modifications to the catheter without increasing the cost of its manufacture.

Other objects and advantages of the invention will become apparent from the following descriptions and drawings.

### BRIEF DESCRIPTION OF THE DRAWINGS

FIG. 1 is a view of a standard triple lumen catheter used in hospital ICUs.

FIG. 2 is a catheter attached to a length of Tygon tubing to test the purge volume of the unmodified device.

FIG. 3 shows a standard triple lumen catheter changed to test a modification of tail internal diameter (I.D.).

FIG. 4 shows a standard catheter with one tail reduced in length from 15 to 3 cm but retaining the usual internal diameter.

FIG. 5 shows how a female Luer connector was modified to reduce the normal internal gap, thereby contributing further to a reduced purge volume.

FIG. 6 shows a doubly modified catheter with a reduced I.D. tail and a zero gap Luer.

FIG. 7 shows a method of measuring the theoretical minimum purge volume.

FIG. 8 shows a triply modified catheter with a shortened tail, a reduced internal diameter for that tail, and a zero gap Luer.

FIG. 9 is a chart showing the results from testing the various catheter modifications.

FIG. 10 shows the configuration of a clinically useful triple lumen catheter made with all the described modifications to give the lowest possible purge volume when monitoring blood glucose.

### DETAILED DESCRIPTION OF THE DRAWINGS

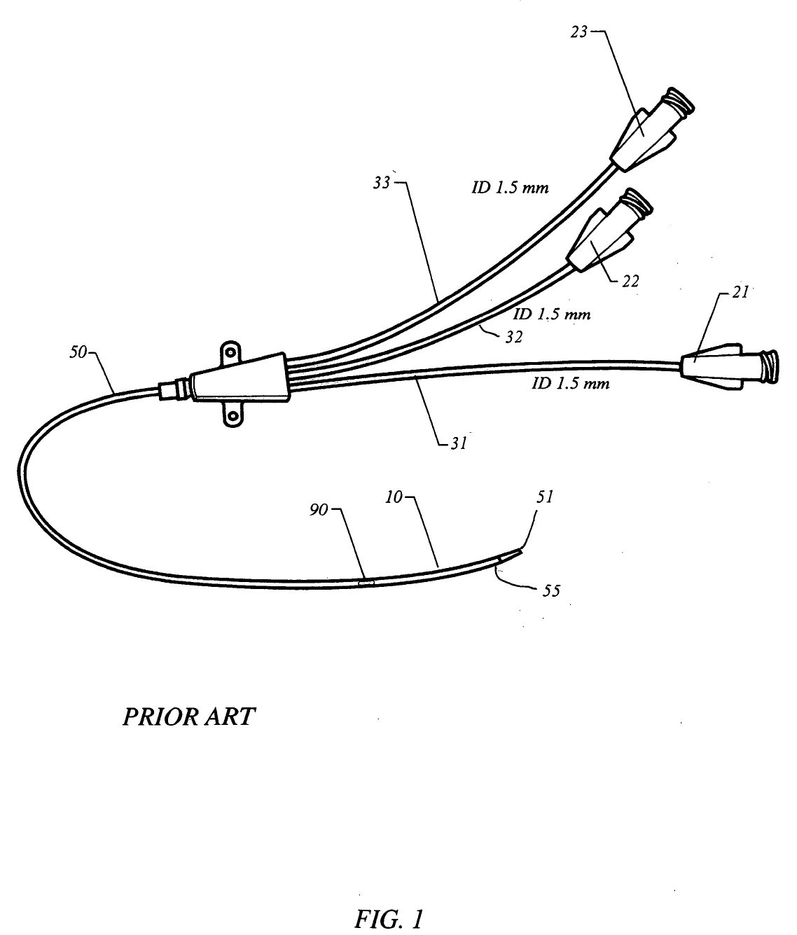

FIG. 1 shows the standard prior art multi-lumen central venous catheter which is commonly used in hospital intensive care units. The inserted portion 50 has a distal end 55 and carries a central lumen with a tip aperture 51 and two side lumens with apertures about 2 and 4 centimeters from the distal end of the catheter. As will be explained in the paragraphs to follow, clinical sampling can be done with accurate results only when the proximal aperture 90 and its lumen are used for obtaining blood samples. The normal internal diameter of all three connecting tubings 31, 32 and 33 (the “tails”) is 1.5 mm. Each connecting tubing carries a female Luer 21,22 and 23.

A standard central venous catheter typically has two 18-gauge (0.75 mm) side lumens and one 16-gauge (1.0 mm) central lumen. Only the side lumens were used in testing these catheters.

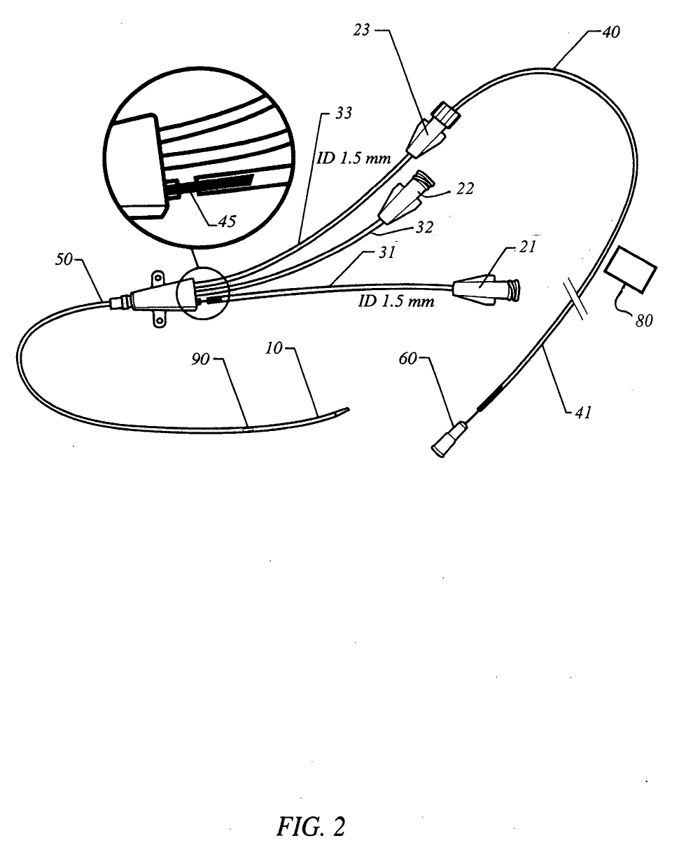

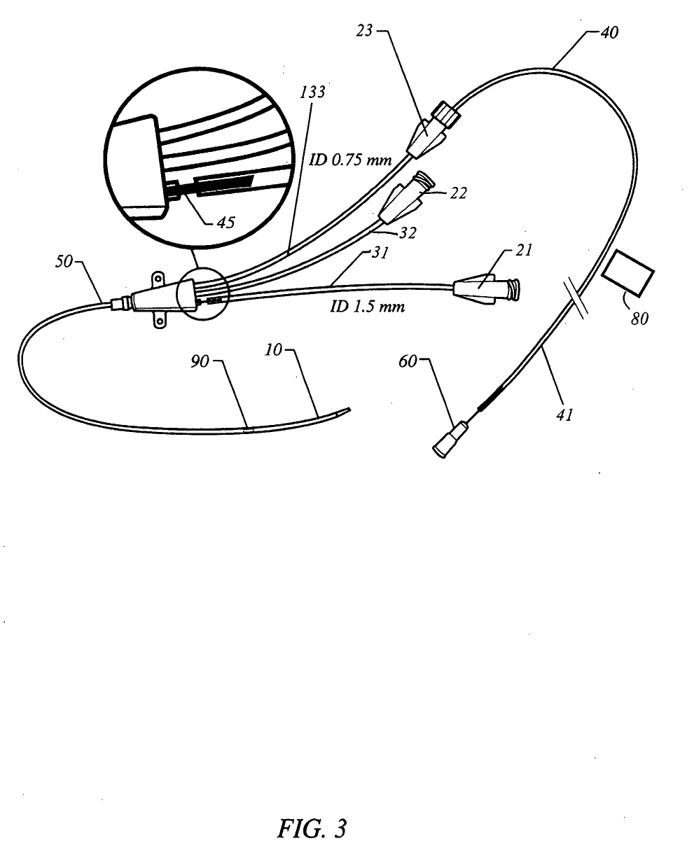

In the test set-up of FIG. 2, the catheter tail 31 is connected to the inserted part 50 of the catheter with a canula 45 made from a #20 needle, seen best in the enlarged view. Fluid can be injected into the proximal end of the one-meter length of 0.75 mm I.D. Tygon tubing 40 through a #20 needle 60 that is inserted into the proximal end 41 of the tubing 40. An I.D. of 0.75 mm was selected for tubing 40 because this size tubing is the smallest diameter that allows for efficient movement of fluid by a peristaltic pump and matches the I.D. of the 18-gage side lumens of a typical central venous catheter. A 5-mL syringe with 3 mL of fluid (not shown) is attached to the needle. Blood is withdrawn until a pure sample is obtained at a point in the line at the approximate position of an optical sensor 80, as described in U.S. patent application Ser. No. 12/231,531. Generally, because of unavoidable mixing of blood and fluid at their interface, blood must be withdrawn to about 50 cm above the Luer fitting 23 when using tubing 40 with an I.D. of 0.75 mm. The purity of the sample is detected with an optical sensor 80, as shown in FIG. 3 of U.S. patent application Ser. No. 12/231,531. The optical sensor 80 sends infrared light across the line where it is received by a photodetector on the opposite side. A pure sample is present when maximum opacity is seen by the detector, indicating that there is no dilution of blood by clear fluid. A blood sample at the location of the optical sensor can then be tested for glucose. Previous tests have shown that from 0.2 to 0.6 mL of blood must be withdrawn to obtain a pure sample, depending on the length of the central venous catheter and the internal diameter of its components.

In the test set-up shown in FIG. 2, after drawing up a blood sample from a vial of blood (not shown), the plunger of the 5-mL syringe 60 is pressed downward, pushing out clear fluid, which moves the blood column out of the central venous catheter and the lower portion of the Tygon tubing 40. The line is purged when fluid coming from the tip 10 of the catheter is seen to be perfectly clear.

In a system wherein blood is drawn up for testing inside a wearable sensor, no residual blood must be left in any part of the line including the central venous catheter. If any residual blood is present when a new sample is withdrawn, an accurate result will not be obtained because the sensor will then be testing both old and new blood. The clear fluid required to clean out the line after a test is called purge fluid and is also used to calibrate the sensor. It is desirable to limit the amount of purge fluid required so as not to overload the patient's circulation and also to keep fluid bag changes to a minimum.

When withdrawing a blood sample in the clinical situation, it is essential that only the so-called proximal lumen 90 of the central venous catheter be used. The proximal lumen aperture (90) is upstream of any introduced IV fluid and therefore not subject to dilutional inaccuracies from fluid infusions.

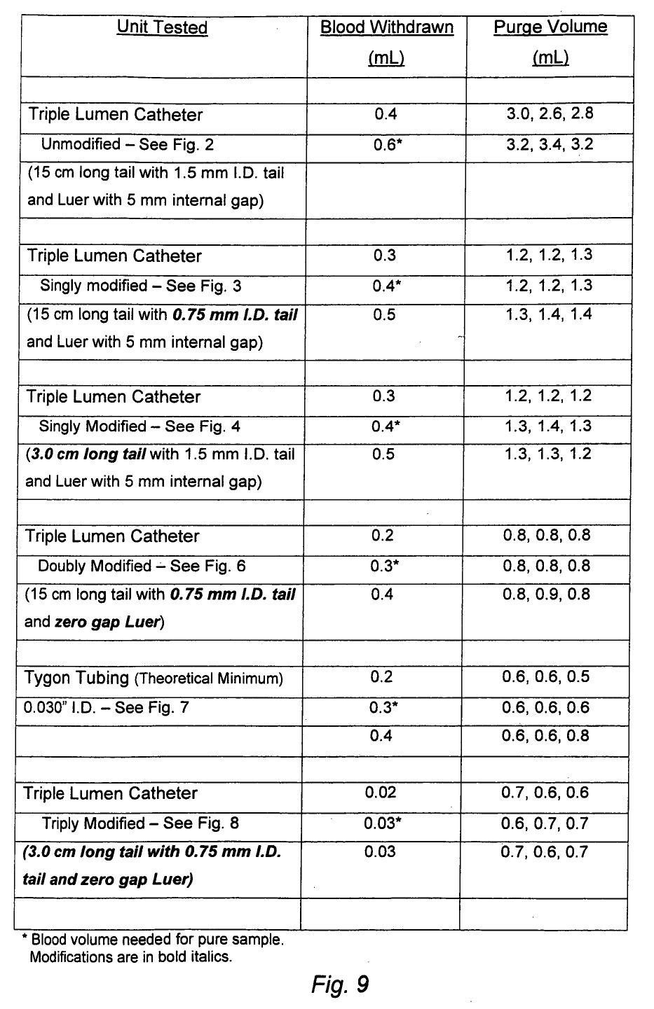

FIG. 2 shows Tygon tubing 40 connected to the unmodified tail 31 of the multi-lumen catheter which has an I.D. of about 1.5 mm, a length of 15 cm and the usual female Luer fitting 21. As shown in the chart of FIG. 9, the volume of fluid needed to purge the line is about 3 mL due to excessive mixing of blood and clear calibration fluid. If about 150 tests are done every 24 hours, or about one every 10 minutes, the amount of purge fluid required is about 450 mL per day. This amount of additional fluid would be an unacceptable addition to the circulation of many patients who might already be over-hydrated.

FIG. 3 shows a modified catheter with the transparent tail 133 of the standard length of 15 cm, but the internal diameter of the tail has been reduced to 0.75 mm by increasing the wall thickness of the tubing. The outer diameter of the tubing is unchanged so that no mold change is needed when manufacturing the catheter. Fluid movement is not restricted because the I.D. of the transparent segment is the same as that of the channel inside the inserted part 50 of the catheter. Purge volume is reduced considerably, from about 3 mL to about 1.2 mL, as shown in the chart of FIG. 9.

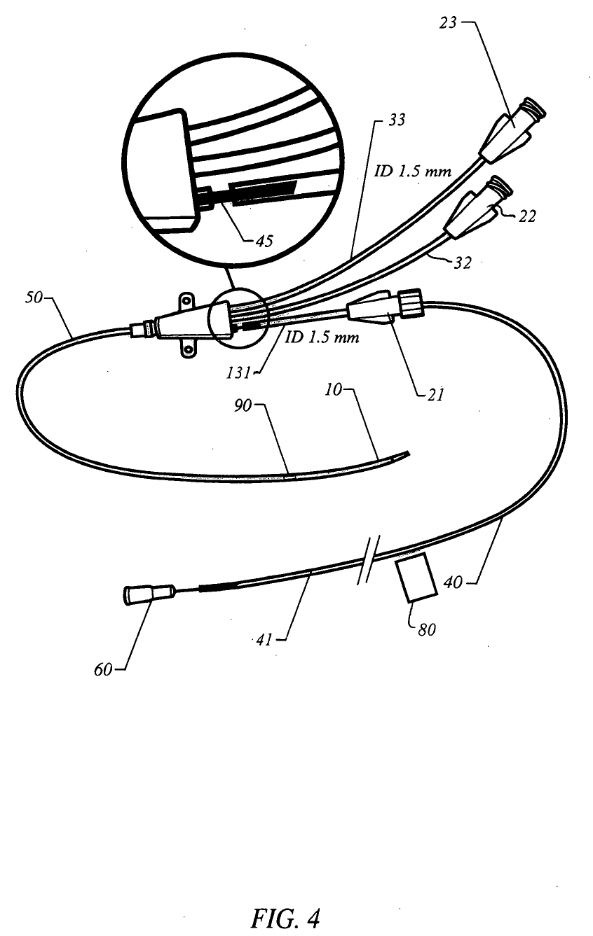

FIG. 4 shows a modified catheter in which the standard I.D. of 1.5 mm was retained inside the connecting tubing 131, but the length of the tubing was changed from 15 to 3 cm. Purge volume was found to be considerably reduced by simply shortening the tubing length. This reduced volume is listed in the chart of FIG. 9.

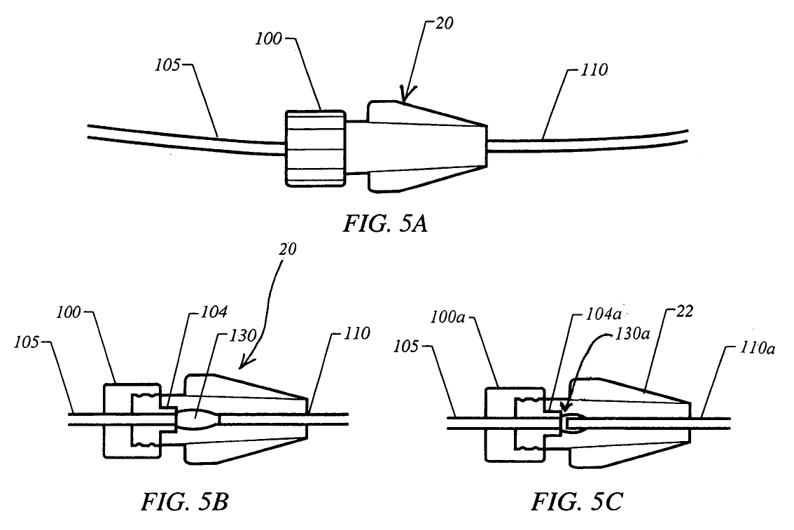

FIG. 5A is a side elevational view of standard Luer connector 20.

FIG. 5B is a cross-section of FIG. 5A and shows that the transparent connecting tubing 110 normally present in the fitting does not touch the tip 104 of the male Luer 100 but instead ends about 5 mm away from it, leaving a gap 130 where blood can collect and cause mixing of blood and clear fluid during purging of the line. This gap is seen in all of the present brands of multi-lumen catheters.

FIG. 5C shows a modified Luer 22 according to the invention. Male Luer plug 100 was first drilled out so that a length of 0.75-mm tubing 105 could be inserted to the tip 104a of the male connector 100a. The tubing 105 was epoxied in place. Next a standard female Luer 20 was drilled out so that its 1.5-mm I.D. tubing 110 could be replaced with tubing 110a having an I.D. of 0.75 mm. To make the Luer fitting shown in FIG. 5C, the tubing 110a was advanced to the tip 104a of the male Luer 100a and then epoxied in place. While a small gap 130a is shown for clarity, in reality this gap is completely eliminated by tightly screwing the two connectors together to give an unbroken fluid column with an internal diameter of 0.75 mm.

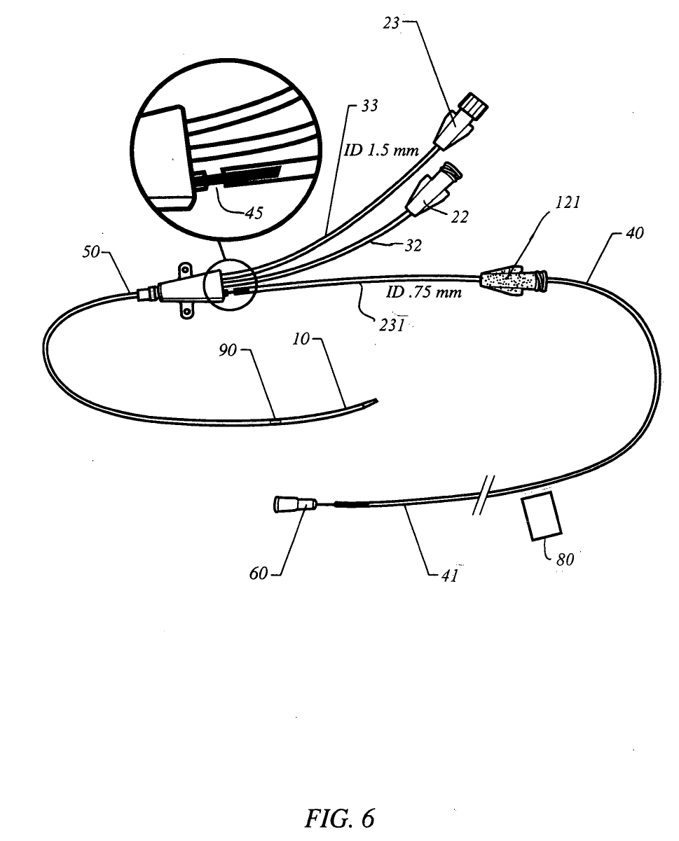

FIG. 6 shows the set-up used for testing another method of doubly modifying a triple lumen catheter. In this version, the zero gap Luer fitting 121, as shown above in FIG. 5C, is paired with a connecting tail 231 with a reduced I.D. of 0.75 mm. Reduced purge volumes are shown in FIG. 9.

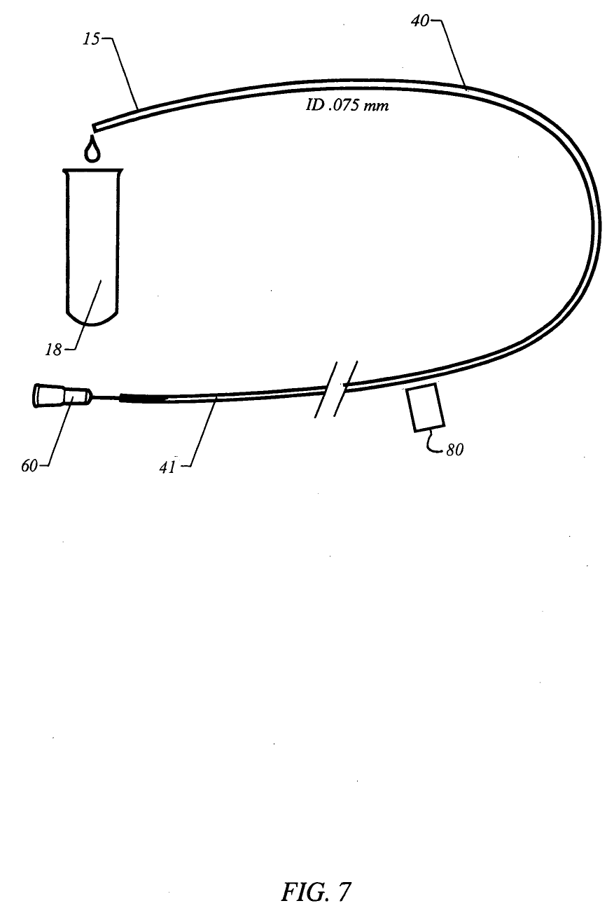

FIG. 7 shows the set-up used to determine the theoretical minimum purge volume. A 4-foot length of plain Tygon tubing 40 has an optical sensor 80 placed about 7 inches from its distal end 15. A needle 60 for a 5-mL syringe is inserted into the tubing at its proximal end 41. The syringe is filled with fluid to 3 mL. Blood from the vial 18 is drawn into the tubing to about 16 cm above the optical sensor, assuring that a pure sample is present for analysis. All factors which could increase the purge volume such as Luer connectors or changes in tubing diameter or shape have been removed. The purge volume required to clear blood from the line, due to unavoidable mixing of blood and fluid, is still from 0.5 to 0.8 mL, which is considered to be the theoretical minimum.

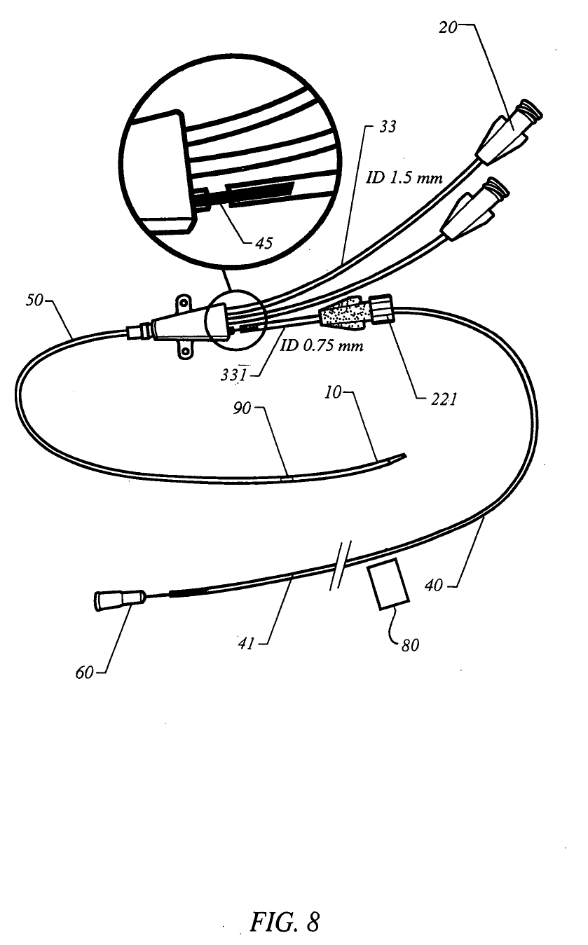

FIG. 8 shows the results of triply modifying a standard central venous catheter (Arrow International) by reducing the internal diameter of the connecting transparent tail 331, by shortening the length of tail 331, and by modifying the female Luer fitting 221 to reduce the internal gap. Each modification contributed to the reduced purge volume.

As seen in the chart of FIG. 9, a central venous catheter with all three modifications shown in FIG. 8 had the lowest purge fluid volume and almost reached the theoretical minimum.

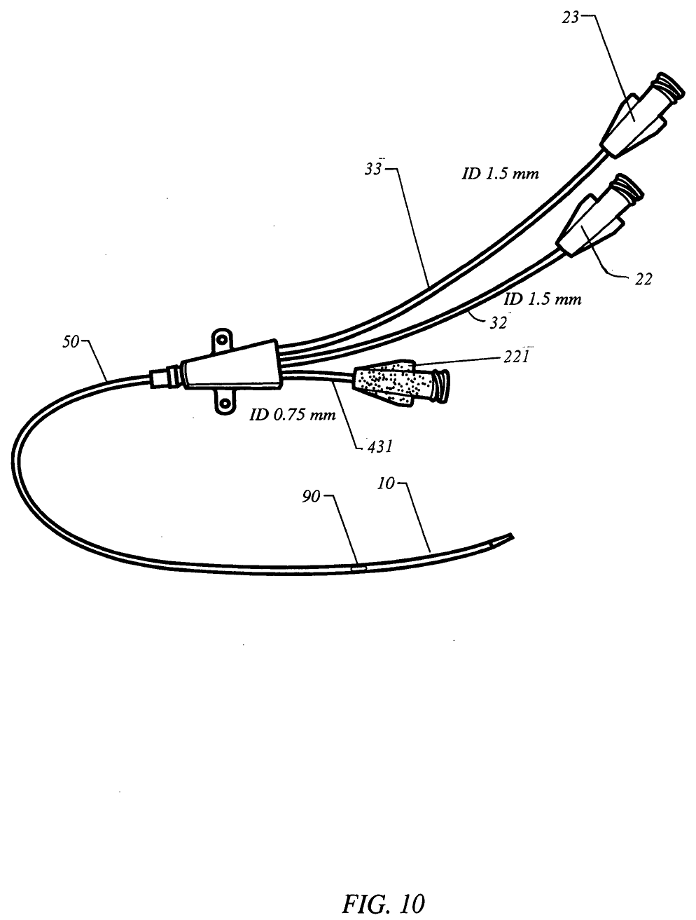

FIG. 10 shows the configuration of a triple lumen catheter made without cannula 41, but with the three modifications previously described. The shortened and reduced diameter “tail” 431 with its gapless Luer connector 221 leads into the side lumen with the most proximal aperture 90. Fluid infusions from lower in the catheter will thus not cause a dilutional inaccuracy in samples withdrawn by testing.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

What is claimed is:

1. A modified multi-lumen catheter with a reduced purge volume comprised of:
one central lumen and two or more side lumens,
lengths of connecting tubing (“tails”) for each of said catheter lumens,
female Luer connectors for each length of connection tubing, and
an internal diameter for at least one of said connecting tubings of less than 1.50 mm.

2. The apparatus of claim 1 wherein at least one length of connecting tubing has an optimum internal diameter of 0.75 mm.

3. A modified multi-lumen catheter with a reduced purge volume comprised of:
one central lumen and two or more side lumens,
lengths of connecting tubing (“tails”) for each of said catheter lumens,
female Luer connectors for each length of connection tubing, and
a length of at least one of the connecting tubings of less than 15 cm.

4. The apparatus of claim 3 wherein at least one of the connecting tubing has an optimum length of about 3 cm.

5. A modified multi-lumen catheter with a reduced purge volume comprised of:
one central lumen and two or more side lumens,
lengths of connecting tubing (“tails”) for each of said catheter lumens,
female Luer connectors for each length of the lengths of said connection tubing, and
a female Luer connector for at least one of the tubings wherein the gap between the end of the connecting tubing and the tip of the joining male Luer is less than 5 mm.

6. The apparatus of claim 5 wherein optimally there is no gap between the end of the connecting tubing and the tip of the joining male Luer.

7. A modified multi-lumen catheter with a reduced purge volume comprised of:
one central lumen and two or more side lumens,
lengths of connecting tubing (“tails”) for each of said catheter lumens,
female Luer connectors for each length of connection tubing, and
at least one length of connecting tubing with an internal diameter of less than 1.5 mm, a length of less than 15 cm, and a gap between the end of the connecting tubing and the tip of the male Luer of less than 5 mm.

8. A modified multi-lumen catheter with a reduced purge volume comprised of:
one central lumen and two or more side lumens,
lengths of connecting tubing (“tails”) for each of said catheter lumens,
female Luer connectors for each length of connection tubing, and
at least one length of connecting tubing with an optimal length of about 3 cm, an optimal internal diameter of about 0.75 mm, and with no gap inside its Luer connector.

9. The apparatus of claim 8 wherein the most proximal catheter aperture is used for the withdrawal of blood samples.
